(19) United States
(12) Patent Application Publication
(10) Pub. No.: US 2026/0258337 A1
(43) Pub. Date: Sep. 3, 2026

(54) MICROCAVITY VESSEL TRANSPORT DEVICES AND SYSTEMS

(71) Applicant: CORNING INCORPORATED, CORNING, NY (US)

(72) Inventors: Thomas Albert Cloutier, Arundel, ME (US); Christopher Bowman Horner, Santa Cruz, CA (US)

(21) Appl. No.: 18/877,725

(22) PCT Filed: Jun. 28, 2023
(86) PCT No.: PCT/US2023/069264

§ 371 Date: Dec. 20, 2024

Related U.S. Application Data
(60) Provisional application No. 63/357,308, filed on Jun. 30, 2022.

(51) Int. Cl. C12M 1/00; C12M 1/32; C12M 3/00

(52) U.S. Cl. CPC C12M 23/52; C12M 23/12; C12M 23/48; C12M 23/54

(57) ABSTRACT

A microcavity vessel transport device is disclosed. In one embodiment, the microcavity vessel transport device may include a base; the base may include a cavity that may receive a microcavity cell culture vessel. The base may also include a plurality of sidewalls defining the cavity and each sidewall may include a bottom wall. The microcavity vessel transport device may also include an arm including a proximal end and a distal end. The proximal end of the arm may be connected to the base and the arm may extend upwards and towards a center of the base, such that the distal end of the arm is positioned over the center of the base.

### CROSS-REFERENCE TO RELATED APPLICATIONS

This application claims the benefit of priority under 35 U.S.C. § 119 of U.S. Provisional Application Ser. No. 63/357,308 filed on Jun. 30, 2022, the content of which is relied upon and incorporated herein by reference in its entirety.

### BACKGROUND

### Field

The present disclosure generally relates to three-dimensional (3D) cell culture and particularly relates to a device for transporting 3D microcavity cell culture vessels.

### Technical Background

Cell culture experts need to transport 3D microcavity vessels, such as plates and flasks, to and from incubators, cell culture hoods, microscopes, and other lab equipment. Due to the large quantity of cells in a 3D culture, and small features of cell culture vessels, it can be extremely difficult to transport such microcavity plates and flasks without generating media movement in the vessel. Such media movement in the microcavity culture vessel results in dislodged spheroids, wherein the spheroids cultured in the microcavity vessel are displaced from the respective wells or cavities. Ultimately, dislodgement or displacement may lead to a loss of uniformity, wherein there is more than one spheroid per microcavity, or loss of spheroids.

Accordingly, a need exists for a device that transports microcavity vessels with minimal displacement of spheroids.

### SUMMARY

According to a first aspect of the present disclosure, a microcavity vessel transport device includes: a base comprising: a cavity, wherein the cavity is configured to receive a microcavity cell culture vessel; and a plurality of sidewalls defining the cavity, each sidewall comprising a bottom wall; and an arm comprising a proximal end and a distal end, wherein the proximal end of the arm is connected to the base and the arm extends upwards and towards a center of the base such that the distal end of the arm is positioned over the center of the base.

A second aspect includes the device of the first aspect, wherein the plurality of sidewalls form a perimeter of the base, and the bottom walls extend a distance inward from the perimeter.

A third aspect includes the device of the first or second aspects, wherein the bottom walls are substantially perpendicular to the plurality of sidewalls.

A fourth aspect includes the device of any of the preceding aspects, wherein the cavity is configured to receive a plurality of microcavity cell culture vessels.

A fifth aspect includes the device of any of the preceding aspects, the base further comprising an adapter removably positioned within the cavity, wherein the adapter narrows a cross-sectional area of the cavity.

A sixth aspect includes the device of any of the preceding aspects, wherein the base further comprises a plurality of standoffs disposed on either of the plurality of sidewalls or the bottom walls.

A seventh aspect includes the device of the sixth aspect, wherein each standoff of the plurality of standoffs forms an “L” shape with one edge disposed on the sidewall and one edge disposed on the bottom wall.

An eighth aspect includes the device of any of the preceding aspects, wherein the distal end of the arm comprises an eyehole extending through the arm.

A ninth aspect includes the device of the eighth aspect, wherein the eyehole is positioned in the arm such that the eyehole is disposed over the center of the base.

An tenth aspect includes the device of any of the preceding aspects, wherein a combined weight of the base and the arm is greater than or equal to 2.5 pounds.

An eleventh aspect includes a microcavity vessel transport device including: a base comprising a plurality of cavities, wherein each of the plurality of cavities is configured to receive a microcavity cell culture vessel; and an arm comprising a proximal end and a distal end, wherein the proximal end of the arm is connected to a center of the base and the arm extends substantially perpendicular from the base.

A twelfth aspect includes the device of the eleventh aspect, wherein the plurality of cavities comprises two or four cavities.

A thirteenth aspect includes the device of the eleventh or twelfth aspects, wherein each of the plurality of cavities is configured to receive a plurality of microcavity cell culture vessels.

A fourteenth aspect includes the device of any of the eleventh through thirteenth aspects, wherein the microcavity cell culture vessel comprises a microcavity flask or a microcavity plate.

A fifteenth aspect includes the device of the fourteenth aspect, wherein the base further comprises an adapter removably positioned in one of the plurality of cavities, wherein the adapter narrows a cross-sectional area of the one of the plurality of cavities.

A sixteenth aspect includes the device of any of the eleventh through fifteenth aspects, wherein a combined weight of the base and the arm is greater than or equal to 2.5 pounds.

A seventeenth aspect includes a microcavity vessel transport system including: a microcavity cell culture vessel; and a microcavity vessel transport device comprising: a base comprising: a cavity, wherein the microcavity cell culture vessel is removably positioned in the cavity; and a plurality of sidewalls defining the cavity, each sidewall comprising a bottom wall; and an arm comprising a proximal end and a distal end, wherein the proximal end of the arm is connected to the base and the arm extends upwards and towards a center of the base, wherein the arm comprises a plurality of eyeholes extending through the arm proximate the distal end of the arm.

An eighteenth aspect includes the system of the seventeenth aspect, further comprising a flexible, compliant holding medium removably attached to at least one of the plurality of eyeholes.

A nineteenth aspect includes the system of the eighteenth aspect, wherein the flexible, compliant holding medium is extends through one of the plurality of eyeholes.

A twentieth aspect includes the system of any of the seventeenth through nineteenth aspects, wherein the microcavity cell culture vessel comprises a microcavity flask or a microcavity plate.

These and other aspects, advantages, and salient features will become apparent from the following detailed description, the accompanying drawings, and the appended claims.

### BRIEF DESCRIPTION OF THE DRAWINGS

FIG. 1 schematically depicts a perspective view of a microcavity vessel transport device, according to one or more embodiments shown and described herein;

FIG. 2 schematically depicts a top-view of the microcavity vessel transport device of FIG. 1, according to one or more embodiments shown and described herein;

FIG. 3 schematically depicts a side-view of the microcavity vessel transport device of FIG. 1, according to one or more embodiments shown and described herein;

FIG. 4 schematically depicts a perspective view of a microcavity vessel transport device, according to one or more embodiments shown and described herein;

FIG. 5 schematically depicts a top-view of the microcavity vessel transport device of FIG. 4, according to one or more embodiments shown and described herein;

FIG. 6 schematically depicts a side-view of the microcavity vessel transport device of FIG. 4, according to one or more embodiments shown and described herein;

FIG. 7 schematically depicts a perspective view of a microcavity vessel transport device, according to one or more embodiments shown and described herein;

FIG. 8 schematically depicts a top-view of the microcavity vessel transport device of FIG. 7, according to one or more embodiments shown and described herein;

FIG. 9 schematically depicts a side-view of the microcavity vessel transport device of FIG. 7, according to one or more embodiments shown and described herein;

FIG. 10 schematically depicts a perspective view of the microcavity vessel transport device of FIG. 1 and a microcavity cell culture vessel, according to one or more embodiments shown and described herein;

FIG. 11 schematically depicts a perspective view of the microcavity vessel transport device of FIG. 4 and the microcavity cell culture vessel, according to one or more embodiments shown and described herein;

FIG. 12A schematically depicts a perspective view of the microcavity vessel transport device of FIG. 7 and microcavity flask in each cavity, according to one or more embodiments shown and described herein;

FIG. 12B schematically depicts a perspective view of the microcavity vessel transport device of FIG. 7 and a plurality of microcavity flasks in each cavity, according to one or more embodiments shown and described herein;

FIG. 13A schematically depicts a perspective view of the microcavity vessel transport device of FIG. 7 with a microcavity plate in each cavity, according to one or more embodiments shown and described herein; and

FIG. 13B schematically depicts a perspective view of the microcavity vessel transport device of FIG. 7 with a plurality of microcavity plates in each cavity, according to one or more embodiments shown and described herein.

### DETAILED DESCRIPTION

In the following description, like reference characters designate like or corresponding parts throughout the several views shown in the figures. It is also understood that, unless otherwise specified, terms such as “top,” “bottom,” “outward,” “inward,” and the like are words of convenience and are not to be construed as limiting terms. Unless otherwise specified, a range of values, when recited, includes both the upper and lower limits of the range as well as any sub-ranges therebetween. As used herein, the indefinite articles “a,” “an,” and the corresponding definite article “the” mean “at least one” or “one or more,” unless otherwise specified. It also is understood that the various features disclosed in the specification and the drawings can be used in any and all combinations.

The term “center” as used herein, refers to a centroid of a geometric figure or plane, that is substantially the centroid, such as within 2 inches, within 1.5 inches, within 1 inch, or even within 0.5 inch of the centroid of the geometric figure or plane.

The term “proximal” designates an end, portion, or end portion of the arm that is intended to be nearer a base and further from a lift point, when the device is being moved by an operator.

The term “distal” designates an end, portion, or end portion of the arm that is intended to be nearer the lift point and further from the base, when the device is being moved by an operator.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

Reference will now be made in detail to various embodiments, examples of which are illustrated in the accompanying drawings.

The microcavity vessel transport devices and systems described herein may include a base that may include a cavity. The cavity may receive a microcavity cell culture vessel. The base of the microcavity vessel transport device may also include a plurality of sidewalls defining the cavity, each sidewall including a bottom wall. An arm including a proximal end and a distal end may also be included, wherein the proximal end of the arm is connected to the base and the arm extends upwards and towards a center of the base such that the distal end of the arm is positioned over the center of the base.

In some embodiments, the base may include a plurality of cavities, such that each of the plurality of cavities may receive the microcavity cell culture vessel. The microcavity vessel transport device is particularly well suited for the effective transportation of a microcavity cell culture vessel housing a large quantity of cells in a 3D culture without dislodging spheroids contained in the microcavity cell culture vessel. The microcavity vessel transport device will be described in more detail herein with specific reference to the appended drawings.

During traditional transport methods, such as an operator manually holding and carrying a microcavity cell culture vessel, liquid cell culture medium shifts/sloshes around a microcavity cell culture vessel due to horizontal acceleration of the microcavity cell culture vessel, such as a microcavity flask or microcavity plate. The microcavity flask or microcavity plate may include microcavities that may house a large array of spheroids within the liquid cell culture medium. An operator may carry or transport the microcavity cell culture vessel. Movement of the operator results in the microcavity walls pushing against the liquid cell culture. Since the liquid cell culture has no fixed shape, the liquid cell culture stores the respective potential energy. When the operator movement stops, the potential energy of the liquid cell culture turns into kinetic energy, thereby resulting in oscillation of the liquid cell culture. Such oscillation is undesirable for the large array of spheroids because the spheroids may become dislodged from the respective microcavities, negatively affecting the health and consistency of the spheroid population.

To mitigate the horizontal forces from dislodging spheroids from their respective microcavities, the devices and systems described herein divert forces to a flexible compliant holding medium such as a string, cord, ribbon, or thin wire, which acts as a pendulum. Thus, the devices and systems described herein improve the handling and transportation of microcavity cell culture vessels, such as large arrays of spheroids within microcavities of microcavity flasks and microcavity plates. The devices and systems incorporate spill-resistant technology for the handling and transportation of microcavity cell culture vessels, thereby maintaining spheroids in place within the respective microcavities.

The devices and systems described herein provide a technical advantage of maintaining the spheroids in their respective microcavities during transport, which allows for cell growth and consistency of cultures with a single spheroid per microcavity. The device and systems described herein may further lead to improved performance, yield, and consistency of 3D cell cultures without an anticipated loss of spheroids from microcavity flasks and microcavity plates during transportation as a result of baffles within the microcavity flasks or microcavity plates, or lack thereof. In addition, with greater spheroid retention in microcavity flasks and microcavity plates, the number of spheroids generated per flask may result in cost savings.

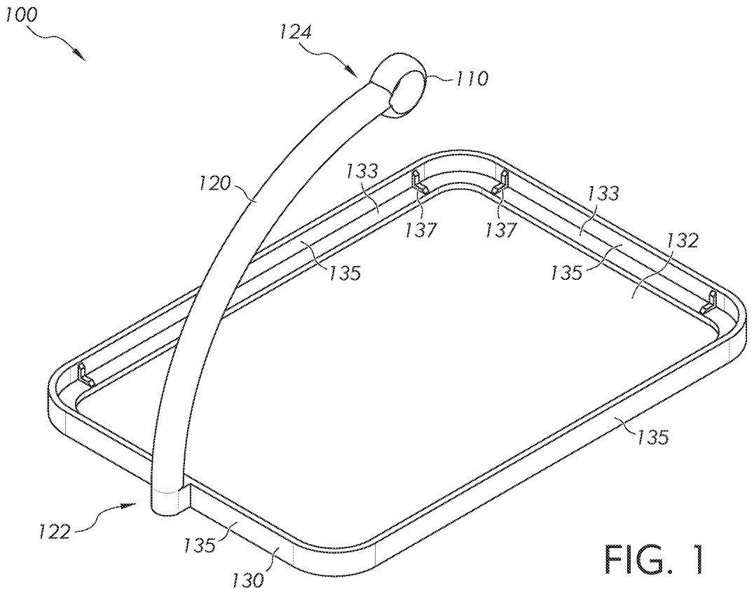

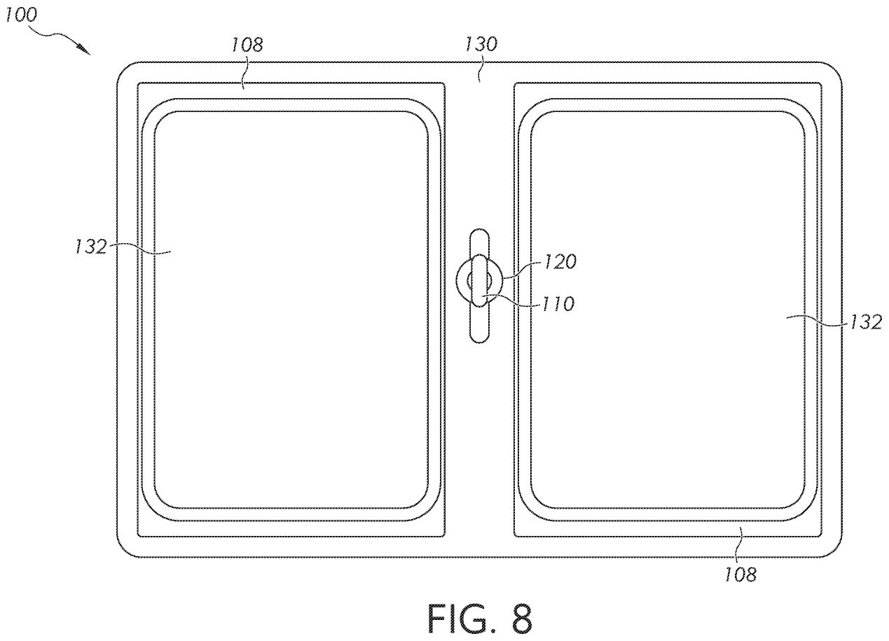

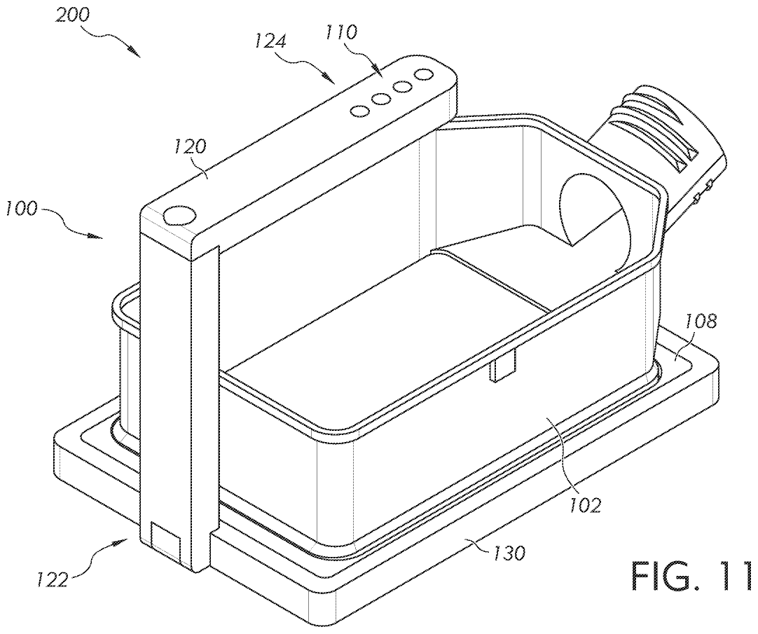

Embodiments of microcavity vessel transport devices are depicted in FIGS. 1-13B. Throughout the drawings, the same reference numerals will be used to refer to the same or like parts. In each of the embodiments of the microcavity vessel transport devices described herein, the microcavity vessel transport devices generally include a base 130 and an arm 120 coupled to the base 130. In each of the embodiments, the base 130 generally defines a cavity 132 for receiving and retaining a microcavity cell culture vessel 102.

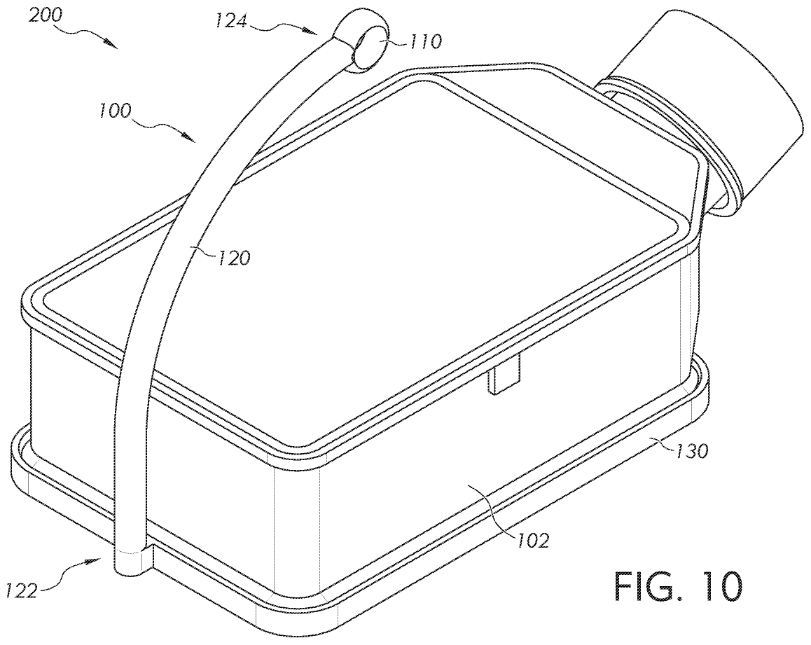

Referring now to FIG. 1, a perspective view of a microcavity vessel transport device 100 is schematically depicted. The microcavity vessel transport device 100 generally comprises a base 130. The base 130 generally comprises a plurality of sidewalls 135 that define a cavity 132. The cavity 132 may be configured to receive a microcavity cell culture vessel 102 (as explained further in the discussion of FIGS. 10-13). Each of the plurality of sidewalls 135 generally comprise a bottom wall 133 to facilitate retaining the microcavity cell culture vessel 102 in the cavity 132. The microcavity vessel transport device 100 also comprises an arm 120. The arm 120 may include a proximal end 122 and a distal end 124.

The cavity 132 may be defined by the plurality of sidewalls 135. The plurality of sidewalls 135 may form a perimeter of the base 130. Moreover, the base may further comprise a plurality of bottom walls 133. The bottom walls 133 may extend inward (i.e., toward a center of the cavity 132) from the perimeter of the base 130 and may be disposed substantially perpendicular to the plurality of sidewalls 135. In embodiments, the bottom walls 133 may extend inward 0.5 inches, 1 inch, 2 inches, 3 inches, 4 inches, or more inward from the perimeter.

The plurality of sidewalls 135 may be formed as one continuous sidewall, such as when the plurality of sidewalls 135 comprise a single piece of material. In other embodiments, the plurality of sidewalls 135 may include two, three, four, or more pieces of material, such that, when the plurality of sidewalls 135 are secured together, the plurality of sidewalls 135 form the perimeter of the base 130. In such embodiments, the plurality of sidewalls 135 may be secured together through adhesives, screws, fasteners, or any other suitable means of securing the plurality of sidewalls 135 to one another.

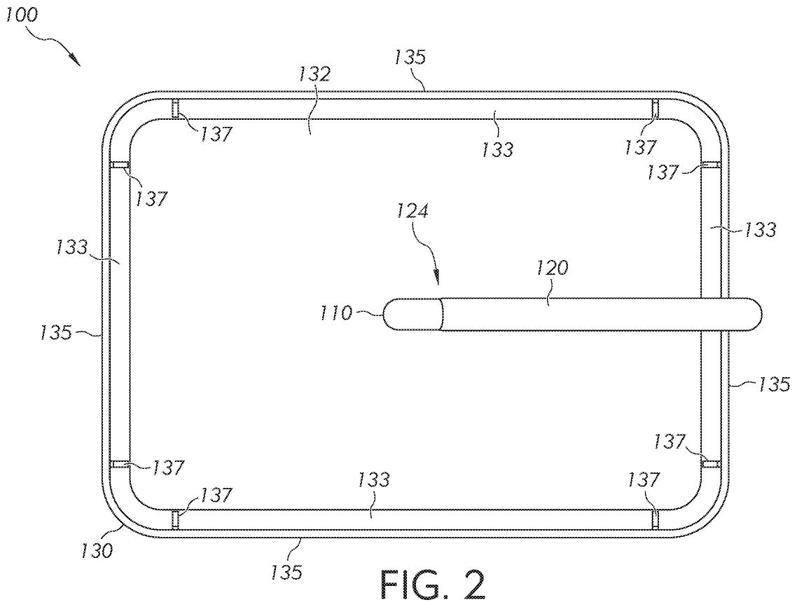

Referring now to FIG. 2, a top-view of the microcavity vessel transport device 100 is schematically depicted. The plurality of sidewalls 135 may include rounded corners. In other embodiments, the plurality of sidewalls 135 may include sharp edged corners, such that the plurality of sidewalls 135 form right angles with respect to one another. The plurality of sidewalls 135 may be formed from polytetrafluoroethylene (Teflon), Polyetherimide (Ultem), polyoxymethylene (Derlin), or any other suitable heat-resistant material.

Furthermore, as depicted in FIG. 1 and FIG. 2, the base 130 may comprise a plurality of standoffs 137. The plurality of standoffs 137 may be disposed on the plurality of sidewalls 135, the bottom walls 133, or both. In embodiments, the base 130 may comprise one, two, three, four, five, six, or more of the standoffs 137 on each of the plurality of sidewalls 135 and the plurality of bottom walls 133. In embodiments, each standoff of the plurality of standoffs 137 may form an “L” shape with one edge disposed on the sidewall 135 and one edge disposed on the bottom wall 133. The plurality of standoffs 137 may contact with the microcavity cell culture vessel 102, such that when the microcavity cell culture vessel 102 is placed within the cavity 132, the microcavity cell culture vessel 102 is raised off of and does not come into contact with either the plurality of sidewalls 135 or the bottom walls 133; instead, the microcavity cell culture vessel 102 is in contact with the plurality of standoffs 137.

In some embodiments, the plurality of standoffs 137 may be integrally formed as part of the plurality of sidewalls 135 and the bottom walls 133. In other embodiments, the plurality of standoffs 137 may be secured to the plurality of sidewalls 135 and the bottom walls 133 through adhesives, screws, fasteners, or any other suitable means of securing the plurality of standoffs 137 to the plurality of sidewalls 135 and the bottom walls 133. The plurality of standoffs 137 may comprise the same or different material than the plurality of sidewalls 135.

As depicted in FIG. 2, an open bottom of the cavity 132 may allow an operator to place a microcavity cell culture vessel 102 directly onto a microscope (for example, when taken out of the incubator or from a cell culture hood) such that the operator is able to visually assess the culture without removing the microcavity cell culture vessel 102 from the microcavity vessel transport device 100. The microcavity cell culture vessel 102 may be supported around the perimeter of the sidewalls 135, the bottom walls 133, or the plurality of standoffs 137, rather than the microcavity cell culture vessel 102 being supported directly on the bottom of the microcavity cell culture vessel 102. This may ensure that the microcavity cell culture vessel 102 stays level when the microcavity cell culture vessel 102 is placed on a surface.

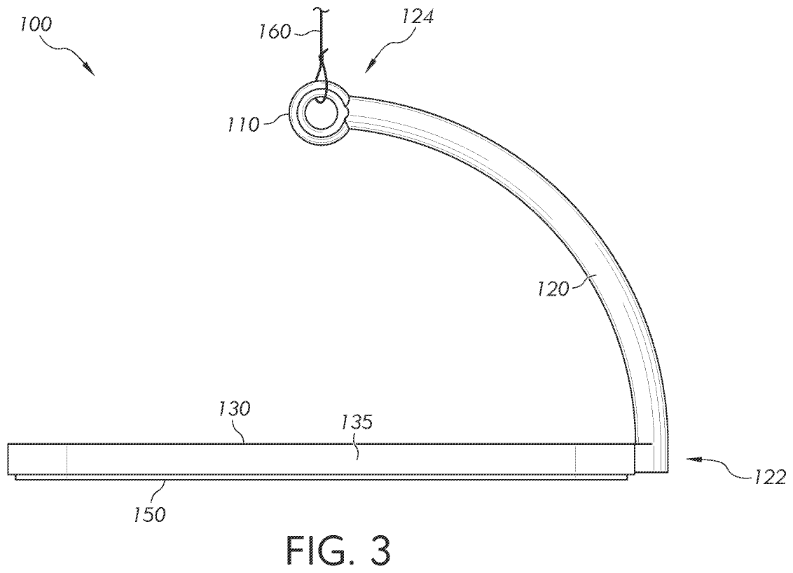

As schematically depicted in a side-view of the microcavity vessel transport device 100 in FIG. 3, the base 130 may include a foot 150 extending downward from the base 130, such that the foot 150 may offset the base 130 from a flat surface; thus, the base 130 is not directly in contact with the surface that the microcavity vessel transport device 100 is placed on when the foot 150 is included. This may prevent the microcavity cell culture vessel 102 from coming into contact with the flat surface, further ensuring that the liquid cell culture medium and, thus, the spheroids contained therein, do not spill/become displaced from the microcavities during transportation of the microcavity cell culture vessel 102.

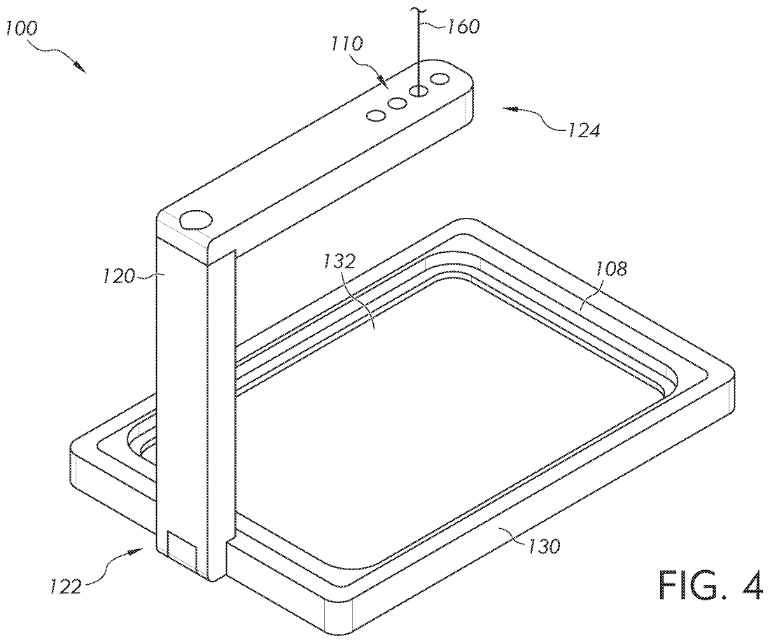

While specific reference is made herein to FIGS. 1-3 in describing the base 130 of the microcavity vessel transport device 100, it should be understood that the base 100 of the microcavity vessel transport devices in each of FIGS. 4-6 and 7-9 may be similarly constructed as the microcavity vessel transport device 100 of FIGS. 1-3.

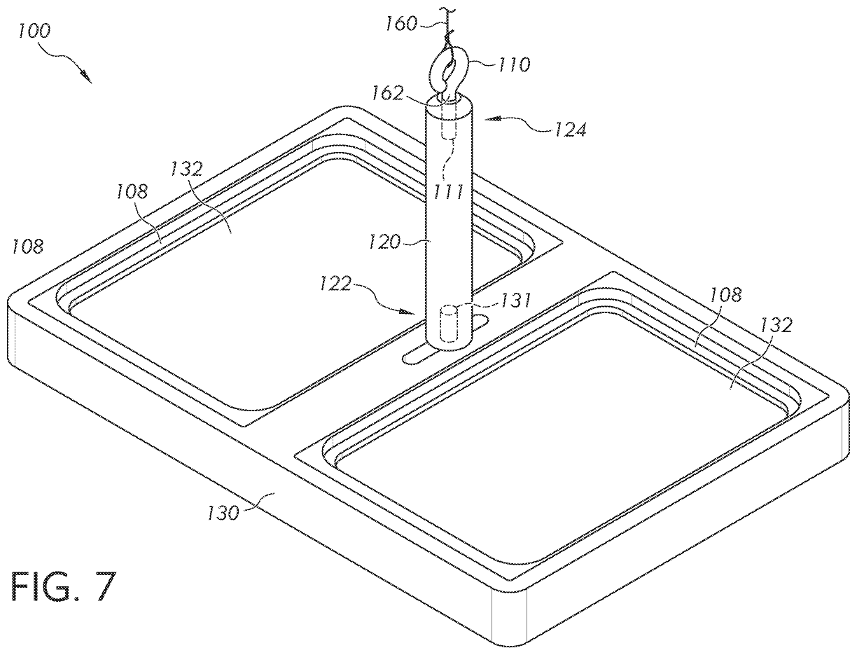

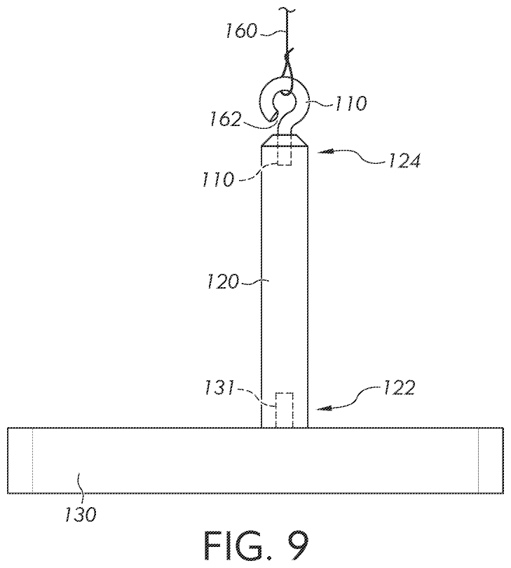

As further depicted in FIG. 3, the arm 120 may include a proximal end 122, which may be connected to the base 130. The proximal end 122 of the arm 120 may be removably connected or permanently connected to the base 130. In some embodiments, the base 130 may include a base extended portion 131 that extends into the proximal end 122 of the arm 120, such that the base extended portion 131 of the base 130 is inserted into a cavity of the arm 120 at the proximal end 122, as depicted in FIG. 7 and FIG. 9 by way of example. In other embodiments, the proximal end 122 of the arm 120 may be part of or otherwise connected to the base 130.

In the embodiments described herein, the arm 120 may extend upwards, away from the base 130. In embodiments, the arm 120 also extends towards a center of the base 130 such that the distal end 124 of the arm 120 is positioned over the center of the base 130, as depicted in FIGS. 1-3 and 4-6. In other embodiments, the arm 120 extends upwards from the center of the base 130, as depicted in the FIGS. 7-9. In the embodiments described herein, the distal end 124 of the arm 120 is positioned approximately over the center of the base 130 so that the microcavity vessel transport device 100 is balanced when the microcavity cell culture vessel 102 is placed within the cavity 132 and the operator moves the microcavity vessel transport device 100. The distal end 124 of the arm 120 is not required to be directly above the center of the base 130. For example, in embodiments, the distal end 124 of the arm 120 may be above the base 130 or at or near the center of the base 130. With such configurations, the microcavity vessel transport device 100 may still be balanced when carrying the microcavity cell culture vessel 102. In other embodiments, the distal end 124 of the arm 120 is positioned over a center of gravity of the microcavity vessel transport device 100, which may or may not be the same position as the center of the base 130.

In the embodiments described herein, the distal end 124 of the arm 120 generally comprises an eyehole 110 extending through the arm 120, such that the eyehole 110 is disposed over the center of the base 130 (or over the center of gravity of the microcavity vessel transport device 100). Although the eyehole 110 is depicted as being at an end of the distal end 124, it is contemplated that the eyehole 110 may be positioned anywhere on the arm 120, so long as the eyehole 110 is disposed over/near the center of the base 130. In embodiments, the eyehole 110 may be part of the distal end 124 of the arm 120, as depicted in FIG. 1 and FIG. 4. In still other embodiments, the eyehole 110 may be removably coupled to the arm 120. As an example, the eyehole 110 may include an eyehole extended portion 111 that extends into the distal end 124 of the arm 120, such that the eyehole extended portion 111 of the eyehole 110 is inserted into a cavity of the arm 120 at the distal end 124, as depicted in FIG. 7 and FIG. 9.

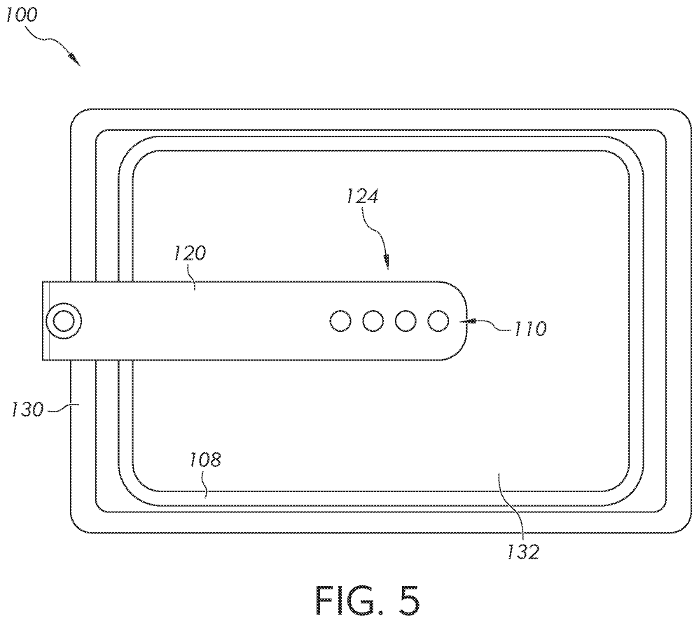

The arm 120 may include a single eyehole 110 as depicted in FIG. 1, or a plurality of eyeholes 110, as depicted in FIG. 4. The eyehole 110 provides a point in which a flexible compliant holding medium 160 may be removably attached to the arm 120 via the eyehole 110. In embodiments, the eyehole 110 may be an enclosed circle, as depicted in FIG. 3 and FIG. 5. In other embodiments, the eyehole 110 may include an eyehole opening 162, such that the flexible compliant holding medium 160 does not have to be untied to be removed from the eyehole 110, as depicted in FIG. 7 and FIG. 9. Instead, the flexible compliant holding medium 160 may simply slide through the eyehole opening 162 to be removed from the eyehole 110. In embodiments in which there are a plurality of eyeholes 110, the flexible compliant holding medium 160 may be removably attached to one or more of the plurality of eyeholes 110.

The flexible compliant holding medium 160 may include a flexible string, cord, ribbon, or other compliant medium. The eyehole 110 may act as the lifting point at which the microcavity vessel transport device 100 is lifted, such that the microcavity vessel transport device 100 may act as a pendulum when the operator makes sudden movements, mitigating the lateral forces that cause sloshing within the a vessel disposed in the microcavity vessel transport device 100. One end of the flexible compliant holding medium 160 may be looped around the eyehole 110 and the opposite end wrapped around the finger or a hand of an operator to facilitate carrying the microcavity vessel transport device 100 without slippage. In other embodiments, the flexible compliant holding medium 160 may be lifted by a machine/robot, rather than a human operator. The flexible compliant holding medium 160 may be untied from the eyehole 110 when the microcavity vessel transport device 100 has reached a desired location.

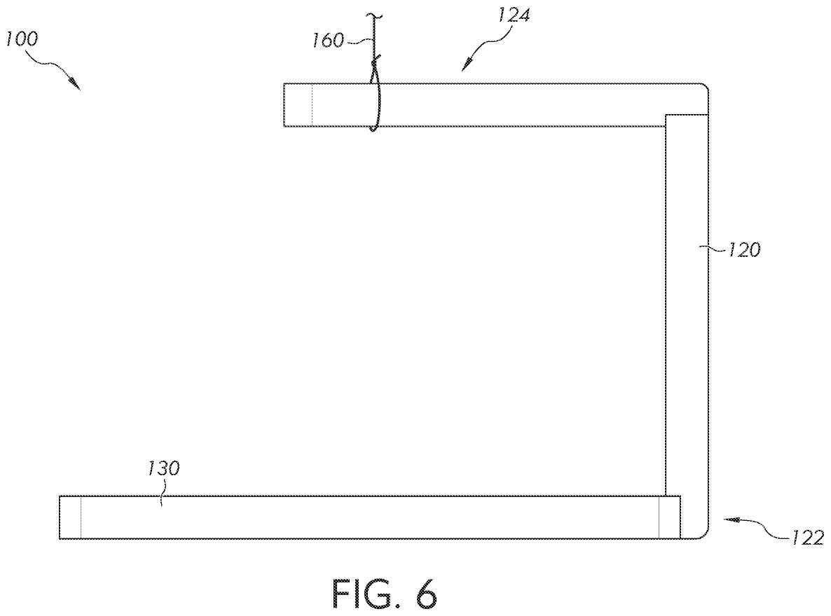

In embodiments, the arm 120 may be curved, form a right angle, or be straight, as depicted in FIGS. 1-3, 4-6, and 7-9, respectively. FIG. 4, FIG. 5, and FIG. 6 schematically depict a perspective view, top-view, and side-view, respectively, of another embodiment of the microcavity vessel transport device 100. As depicted in FIG. 5, the arm 120 may form a right angle, such that the portion of the arm 120 including the proximal end 122 of the arm 120 is substantially perpendicular to the base 130, while the portion of the arm 120 including the distal end 124 of the arm 120 is substantially parallel to the base 130. The length of the portion of the arm 120 including the proximal end 122 may be configured with a sufficient length such that the cavity 132 may receive a plurality of microcavity cell culture vessels 102 stacked on top of one another within the cavity 132.

In the embodiments described herein, a combined weight of the base 130 and the arm 120 may be greater than or equal to 1.0 pounds, greater than or equal to 1.5 pounds, greater than or equal to 2.0 pounds, greater than or equal to 2.5 pounds, greater than or equal to 3.0 pounds, or greater than or equal to 3.5 pounds. This may provide stability to the liquid cell culture medium and the spheroids contained therein when the microcavity vessel transport device 100 is moved by an operator. Moreover, a greater weight of the base 130 and the arm 120 may prevent tipping of the microcavity cell culture vessel 102 when some of the liquid cell culture medium begins to move to one side of the microcavity cell culture vessel 102. Generally, in the embodiments described herein, the combined weight of the base 130 and the arm 120 is greater than 3 times the maximum load of the plurality of microcavity cell culture vessels 102 and the liquid cell culture medium placed within the cavity 132, which may depend on a length of the arm 120 (discussed further herein).

In particular, the liquid cell culture medium may shift to one side of the microcavity cell culture vessel 102 when the operator stops moving or makes a sudden movement. When the combined weight of the base 130 and the arm 120 are less than or close to the weight of the liquid cell culture medium and the microcavity cell culture vessel 102, the microcavity vessel transport device 100 may tip in the direction that the liquid cell culture medium has shifted. This may result in further shifting of the liquid cell culture medium, resulting in spillage of the liquid cell culture medium and displacement of the spheroids. On the other hand, when the combined weight of the base 130 and the arm 120 is large enough, shifting of the liquid cell culture medium will not result in tipping of the microcavity vessel transport device 100 because the weight of the base 130 and the arm 120 may counteract any shift in weight from movement of the liquid cell culture medium.

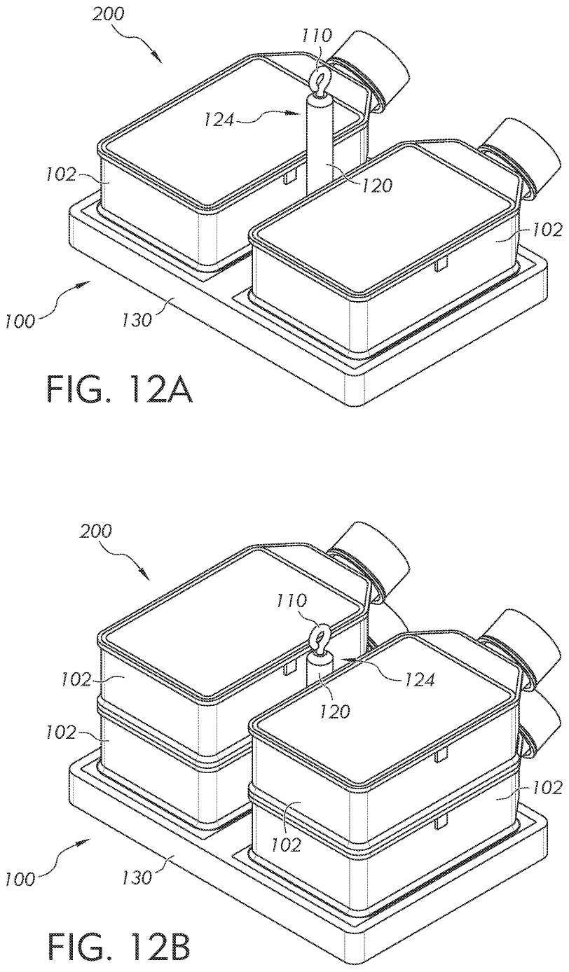

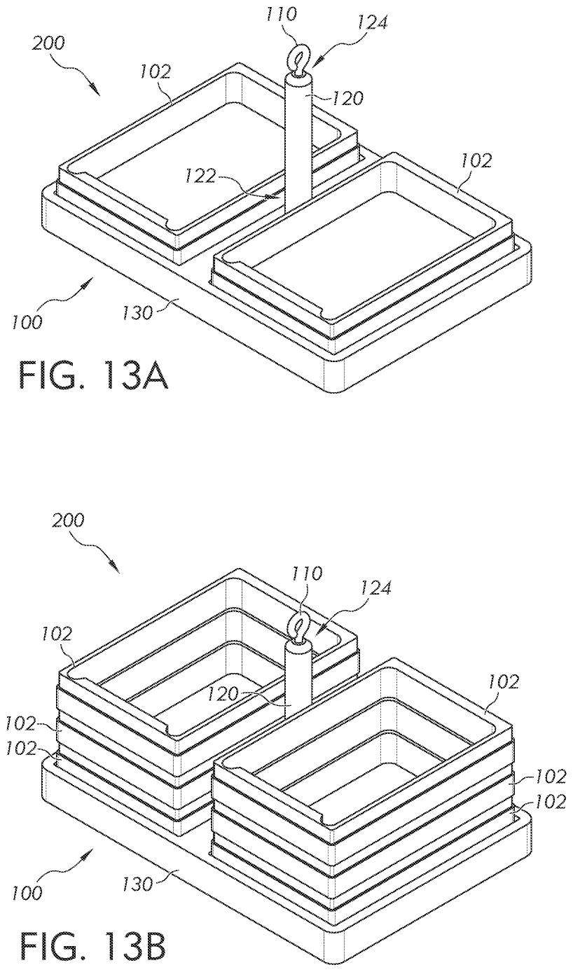

In embodiments, the microcavity cell culture vessel 102 may include a microcavity flask 104 or a microcavity plate 106, depicted in FIG. 12 and FIG. 13, respectively. The microcavity flask 104 and the microcavity plate 106 may each house the liquid cell culture medium and the spheroids. In embodiments, the microcavity flask may generally comprise a 12K Bulk Spheroid flask, or any other suitable flask. The microcavity flask 104 or microcavity plate 106 may include a baffle within the inner volume of the microcavity flask to prevent undesired sloshing of the liquid cell culture medium. However, the baffle may present challenges such as wicking and creating a meniscus that leads to spheroid loss near the baffle feature. The baffles may be unnecessary in light of the current disclosure due to mitigation of sloshing through the microcavity vessel transport device 100.

Various designs of microcavity flasks 104 and microcavity plates 106 may vary in cross-sectional area. As such, the microcavity flask 104 and the microcavity plate 106 may not each securely fit within the sidewalls 135 of the cavity 132. As schematically depicted in FIG. 4 and FIG. 5, an adapter 108 may be removably positioned within the cavity 132 to account for the differences in size between different microcavity flasks 104 and the microcavity plates 106. The adapter 108 may adjust a cross-sectional area of the cavity 132 to accommodate microcavity plates and/or microcavity vessels of different designs or sizes. For example, a microcavity plate 106 of a first design may fit within the cavity 132 when the adapter 108 is not within the cavity 132 and the microcavity flask 104 of a different design may fit within the cavity 132 when the adapter 108 is placed within the cavity 132. As an example, the adapter 108 may narrow the cross-sectional area of the cavity 132 or alter the shape of the cavity 132 so that petri dishes or other vessels may fit within the cavity 132. In embodiments, the adapter 108 may be made of polytetrafluoroethylene (Teflon), Polyetherimide (Ultem), polyoxymethylene (Derlin), or any other suitable material. It should be understood that any of the embodiments of the microcavity vessel transport device 100 described herein may include a removable adapter 108 to facilitate receiving microcavity plates and/or microcavity vessels of different designs or sizes.

Referring now to FIG. 7, in embodiments, the base 130 may include a plurality of cavities 132, such that each of the plurality of cavities 132 may receive the microcavity cell culture vessel 102. As an example, FIG. 7 depicts an embodiment including two cavities in the base 130. However, it is contemplated that there may be three, four, five, six, or more of the plurality of cavities 132 in the base 130. Each of the plurality of cavities 132 may receive one microcavity cell culture vessel 102. FIG. 12A and FIG. 13A depict each of the plurality of cavities 132 receiving one microcavity flask and one microcavity plate, respectively. In other embodiments, each of the plurality of cavities 132 may receive a plurality of microcavity cell culture vessels 102. For example, FIG. 12B and FIG. 13B depict each of the plurality of cavities 132 receiving a plurality of microcavity flasks and a plurality of microcavity plates, respectively.

Each of the plurality of cavities 132 may receive the same number of the microcavity cell culture vessels 102, as depicted in FIG. 12 and FIG. 13. In other embodiments, each of the plurality of cavities 132 may receive a different number of the microcavity cell culture vessels 102. As an example, one of the cavities 132 may receive 1 microcavity cell culture vessel 102 and another of the plurality of cavities 132 may receive 2 of the microcavity cell culture vessels 102. As discussed hereinabove, the weight of the base 130 and the arm 120 may offset an uneven/shifting weight of the microcavity cell culture vessels 102, such that the base 130 remains substantially level even when an uneven number of the microcavity cell culture vessels 102 are in the plurality of cavities 132.

Each of the plurality of cavities 132 may also include an adapter 108, as described herein. The adapter 108 may allow for the plurality of cavities to receive the microcavity flask (as depicted in FIG. 12) or the microcavity plate (as depicted in FIG. 13). In other embodiments, one of the plurality of cavities 132 may receive the microcavity plate, while the other of the plurality of cavities 132 may receive the microcavity flask through the use of the adapter 108.

When there are two or more cavities 132 in the base 130, the proximal end 122 of the arm 120 may be connected to the center of the base 130 and the arm 120 may extend substantially perpendicular from the base 130, as schematically depicted in FIGS. 7-9. As such, the eyehole 110 at the distal end 124 of the arm 120 may be above the center of the base 130.

In the embodiments depicted in FIGS. 7-9, the arm 120 may extend 6 inches, 8 inches, 1 foot, two feet, or more above the base 130. In some embodiments, the length of the arm 120 may be based on the maximum number of the plurality of microcavity cell culture vessels 102 permitted to be carried by each cavity 132 of the microcavity vessel transport device 100. For example, if the microcavity flask is 4 inches in thickness and the arm 120 extends 8 inches above the base 130, each cavity 132 may only carry 2 of the microcavity flasks. Alternatively, if the microcavity flask is 4 inches in thickness and the arm 120 extends a foot above the base 130, each cavity 132 may carry 3 of the microcavity flasks. The same concept may be applied for the maximum number of microcavity plates permitted to be carried by each cavity 132.

Also contemplated is a microcavity vessel transport system 200, as depicted in FIGS. 10-13. In these embodiments, the microcavity vessel transport system 200 may include the microcavity cell culture vessel 102 and the microcavity vessel transport device 100 as described herein. Aforementioned features of the microcavity cell culture vessel 102 and the microcavity vessel transport device 100 may be combined together in any suitable manner.

It should now be understood that the microcavity vessel transport devices and systems with a base and an arm extending toward a center of the base described herein allow for operators to transport the microcavity vessel transport device with minimal shifting of the liquid cell culture medium or spheroid loss. It should also be understood that the microcavity vessel transport devices with a plurality of cavities may allow for the transportation of the plurality of microcavity cell culture vessels at once. These characteristics make the microcavity vessel transport devices and systems well suited for operators transporting multiple microcavity cell culture vessels to and from incubators, cell culture hoods, microscopes, and other lab equipment.

While typical embodiments have been set forth for the purpose of illustration, the foregoing description should not be deemed to be a limitation on the scope of the disclosure or appended claims. Accordingly, various modifications, adaptations, and alternatives may occur to one skilled in the art without departing from the spirit and scope of the present disclosure or appended claims.

1. A microcavity vessel transport device comprising:
a base comprising:
a cavity, wherein the cavity is configured to receive a microcavity cell culture vessel; and
a plurality of sidewalls defining the cavity, each sidewall comprising a bottom wall; and
an arm comprising a proximal end and a distal end, wherein the proximal end of the arm is connected to the base and the arm extends upwards and towards a center of the base such that the distal end of the arm is positioned over the center of the base.

2. The microcavity vessel transport device of claim 1, wherein the plurality of sidewalls form a perimeter of the base, and the bottom walls extend a distance inward from the perimeter.

3. The microcavity vessel transport device of claim 1, wherein the bottom walls are substantially perpendicular to the plurality of sidewalls.

4. The microcavity vessel transport device of claim 1, wherein the cavity is configured to receive a plurality of microcavity cell culture vessels.

5. The microcavity vessel transport device of claim 1, the base further comprising an adapter removably positioned within the cavity, wherein the adapter narrows a cross-sectional area of the cavity.

6. The microcavity vessel transport device of claim 1, wherein the base further comprises a plurality of standoffs disposed on either of the plurality of sidewalls or the bottom walls.

7. The microcavity vessel transport device of claim 6, wherein each standoff of the plurality of standoffs forms an “L” shape with one edge disposed on the sidewall and one edge disposed on the bottom wall.

8. The microcavity vessel transport device of claim 1, wherein the distal end of the arm comprises an eyehole extending through the arm.

9. The microcavity vessel transport device of claim 8, wherein the eyehole is positioned in the arm such that the eyehole is disposed over the center of the base.

10. The microcavity vessel transport device of claim 1, wherein a combined weight of the base and the arm is greater than or equal to 2.5 pounds.

11. A microcavity vessel transport device comprising:
a base comprising a plurality of cavities, wherein each of the plurality of cavities is configured to receive a microcavity cell culture vessel; and
an arm comprising a proximal end and a distal end, wherein the proximal end of the arm is connected to a center of the base and the arm extends substantially perpendicular from the base.

12. The microcavity vessel transport device of claim 11, wherein the plurality of cavities comprises two or four cavities.

13. The microcavity vessel transport device of claim 11, wherein each of the plurality of cavities is configured to receive a plurality of microcavity cell culture vessels.

14. The microcavity vessel transport device of claim 11, wherein the microcavity cell culture vessel comprises a microcavity flask or a microcavity plate.

15. The microcavity vessel transport device of claim 14, wherein the base further comprises an adapter removably positioned in one of the plurality of cavities, wherein the adapter narrows a cross-sectional area of the one of the plurality of cavities.

16. The microcavity vessel transport device of claim 11, wherein a combined weight of the base and the arm is greater than or equal to 2.5 pounds.

17. A microcavity vessel transport system comprising:
a microcavity cell culture vessel; and
a microcavity vessel transport device comprising:
a base comprising:
a cavity, wherein the microcavity cell culture vessel is removably positioned in the cavity; and
a plurality of sidewalls defining the cavity, each sidewall comprising a bottom wall; and
an arm comprising a proximal end and a distal end, wherein the proximal end of the arm is connected to the base and the arm extends upwards and towards a center of the base, wherein the arm comprises a plurality of eyeholes extending through the arm proximate the distal end of the arm.

18. The microcavity vessel transport system of claim 17, further comprising a flexible, compliant holding medium removably attached to at least one of the plurality of eyeholes.

19. The microcavity vessel transport system of claim 18, wherein the flexible, compliant holding medium is extends through one of the plurality of eyeholes.

20. The microcavity vessel transport system of claim 17, wherein the microcavity cell culture vessel comprises a microcavity flask or a microcavity plate.
